(19) United States
(12) Patent Application Publication
(10) Pub. No.: US 2018/0116246 A1
(43) Pub. Date: May 3, 2018

(54) PROCESS FOR MANUFACTURING AN ALKALI GRANULATED ADDITIVE FOR ANIMAL FEED AND ALKALI GRANULATED ADDITIVE FOR ANIMAL FEED

(71) Applicant: CJ DO BRASIL INDUSTRIA E COMERCIO DE PRODUTOS ALIMENTICIOS LTDA., Piracicaba (BR)

(72) Inventors: Cezar Yoo Geun Shin, Piracicaba (BR); Hae Ri Kim, Piracicaba (BR); Paulo Henrique de Agostino Paschoalino, Campinas (BR)

(73) Assignee: CJ DO BRASIL INDUSTRIA E COMERCIO DE PRODUTOS ALIMENTICIOS LTDA., Piracicaba (BR)

(21) Appl. No.: 15/566,878

(22) PCT Filed: Apr. 14, 2016
(86) PCT No.: PCT/BR2016/050081

§ 371 Date: Oct. 16, 2017

(30) Foreign Application Priority Data
Apr. 17, 2015 (BR) 102015008762-4

(51) Int. Cl. A23K 10/12; A23K 20/142; A23K 40/10; C12P 13/08

(52) U.S. Cl. CPC A23K 10/12; C12P 13/08; A23K 40/10; A23K 20/142

(57) ABSTRACT

The invention refers to an alkali granulated additive for animal feed with high concentration of lysine, but especially low ammonium content. More particularly, said alkali granulated additive has pH within the range between 8 and 10, including a lysine concentration between 70% and 85% by weight and having a nearly negligible content of ammonium ions which the molar relation follows the condition below:

$$\frac{[ {NH}_{4}^{+} ]}{[ L Lys ]} < {10}^{- 2}$$

being obtained by a process wherein, during the fermentation step inside fermentation tanks, concentration levels of ammonium ions in the solution are controlled by monitoring the rate of ammoniacal nitrogen (AN) throughout processing and fermenting culture medium, so to keep said content of ammoniacal nitrogen (AN) around 100 mg/l until it is close to the end of the carbon source in the culture medium is detected and, subsequently, promote a controlled reduction of the content of the ammoniacal nitrogen (AN) until it reaches amounts between 50 mg/l and 10 mg/l, which will be controlled and monitored until the end of the carbon source in the culture medium.

### CROSS-REFERENCE TO RELATED APPLICATION

This application claims the benefit of the PCT Application No. PCT/BR2016/050081, filed on Apr. 14, 2016, which claims the benefit of Brazil Patent Application No. 102015008762-4, filed on Apr. 17, 2015, the disclosure of which is incorporated herein by reference in its entirety.

### FIELD OF THE INVENTION

The present invention refers to an alkali granulated additive for animal feed with high concentration of lysine, but especially low ammonium content.

Particularly, said alkali granulated additive for animal feed in accordance to the present invention is obtained by means of manufacturing processes constituted by technical and functional features, especially developed to reach a lysine-based granulated additive for animal feed with high concentration of lysine and high pH which, besides disclosing a product with low hygroscopicity, has a substantially reduced ammonium content.

The process for manufacturing a granulated additive for animal feed in accordance to the present invention manages to simplify production lines, but especially to reach a substantial reduction in manufacturing costs not affecting physical and chemical properties of those products, consequently enabling their safe handling and eliminating animal rejection risks due to undesirable odors and the quality of this additive.

### BACKGROUND OF THE INVENTION

According to the state of the art, and as the person skilled in the art should appreciate, the lysine is an amino acid particularly intended for the production of additives for animal feed, which the main purpose is to improve the digestive system of animals, allowing to reach feeds with better digestion ability with direct consequences to the costs involved in animal breeding.

As it is well known in the art, lysine is produced by a biological fermentation process, wherein microorganisms such as aerobic bacteria consume monosaccharides, releasing lysine. More usually nowadays, such microorganisms are typically Corynebacteria or Escherichia coli, and said biomass may vary according to interests and availability, but it should be a carbon-rich source such as sugar cane, sugar beets or any other source containing gross sugar, glucose, sucrose or molasses.

As a result of that fermentation step, we obtain a fermentation broth containing lysine, also simply called broth with lysine. Besides lysine released by microorganisms, said fermentation broth is constituted by other elements and substances, including microorganisms/cells themselves, among other impurities.

After the fermentation step is concluded, the current state of the art for production of additives for animal feed may be, basically, subdivided into two more traditional methods to processing said fermentation broth containing lysine. In generally, more conventional forms for treating, refining, handling and obtaining an additive for animal feed as known in the state of the art are: the crystallization, from which we obtain lysine salt, better known as lysine-HCl; and the granulation, resulting in the so-called granulated lysine.

More particularly, in the crystallization process for manufacturing additives for animal feed, said fermentation broth with lysine is initially submitted to filtration to take off microorganisms and cells, being subsequently submitted to a separation step by means of the so-called ionic exchange as performed by handling pH with the introduction of acids and bases to adhere the lysine to ionic resins, being released afterwards.

As a result of processing involving ionic exchange and later evaporation, a liquid is generated containing high concentration of lysine, herein simply designated as a concentrated liquid, which is submitted to an acidification step by introducing hydrochloric acid, which the purpose is to provide the precipitation of amino acid.

Just as a clarification, in the present text, terms and expressions such as “concentrated liquid”, “purified liquid”, “pure lysine”, “purified lysine”, “liquid lysine” and “concentrated lysine” should be understood as synonyms, since they are related to the liquid containing high concentration of lysine after due processes, such as the ionic exchange and evaporation.

In the precipitation step, the concentrated liquid with hydrochloric acid passes through a separation step, a new concentration and then a drying step. At the end of those steps, we can reach additives for animal feed as a product, in crystal form with high lysine concentration, i.e. a lysine salt, commercially known as Lysine-HCl.

Currently, the crystallization process for manufacturing additives for animal feed is probably the one that can reach higher concentration, thus reaching up to about 80% by weight of lysine, wherein the other 20% represent chloride.

As the person skilled in the art should appreciate, this kind of processing uses a substantial amount of acids, such as hydrochloric acid and sulfuric acid, considering the need to change pH in the fermentation broth to promote lysine precipitation and also the preservation of raw material before the purification step. The use of acids increases molecular weight, thus limiting the concentration of pure lysine in the final product. In other words, the excessive use of acids ends up by affecting and occupying the space which could be used for lysine in the molecular structure of the salt.

One of the main inconveniences of that crystallization process to produce additives for animal feed refers to the involved costs. More particularly, it is possible to note that, to refine the fermentation broth with lysine, substantial investments are required with equipment, as well as high costs with resins for ionic exchange. Furthermore, it generates very damaging effluents requiring special care and treatment, such as cell concentrate, ammonium sulfate and other nitrogenated compounds, besides requiring other chemical products in the purification process, thus increasing the final costs of the additive for animal feed.

In view of these inconveniences, particularly related to high complexity and the costs for processing and obtaining lysine-based additives for animal feed such as lysine salts or lysine-HCl, a process for concentration and granulation of the fermentation broth with lysine has been developed. More specifically, it is possible to conclude that it would be possible to proceed by handling and drying the fermentation broth itself, without requiring the refining steps, especially acidification and precipitation, which brought in high costs for the production of additives for animal feed in accordance to the state of the art. As an example, documents EP 1424012B1, U.S. Pat. No. 7,883,878B2 and EP 582101A1 disclose processes for manufacturing lysine-based animal feed and supplements using the concentration and granulation process of the fermentation broth.

In this concentration and granulation process, the fermentation broth is concentrated and dried by various processes such as drying with a spray drier, fluid bed and by pelletization or granulation by agglomeration, wherein, more particularly, said lysine-containing fermentation broth is sprayed over said solid particle and subsequently dried; spraying and drying of the fermentation broth over said solid particle are subsequently repeated, and said cycle of broth application and drying is repeated numerous times so to increase the concentration level of lysine until forming the granulated lysine particles.

Despite the granulation process presents substantial cost reduction and complexity of equipment and processing, as the person skilled in the art should appreciate, said process discloses other inconveniences, particularly regarding handling and standardization of the final product. This is because, by the nature itself of lysine synthesis during the fermentation step, it would be impossible to reach a standardized fermentation broth in relation to the lysine concentration. In other words, lysine concentration in the fermentation broth may change according to microorganism activities, the quantity and quality of the available biomass, and thus fermentation broths are produced with different lysine concentrations and, for this reason, we cannot reach a standardized granulated lysine, i.e. it is very difficult to reach a standardized final product, which is commercially not very favorable.

With the purpose to try to reach a standardized final product, thus commercially better accepted, the manufacturers of additives for animal feed have decided to adopt alternative methods to reduce the content of lysine in the fermentation broth, thus managing to standardize broth concentration to, consequently, produce additives for animal feed with standardized lysine content.

The problem in these methods for reducing lysine content is the addition of acids, salts and cells to reach the reduction and keep a constant lysine concentration. Therefore, as a consequence of the existence of those other substances, the final product as obtained cannot reach a high lysine concentration. In other words, the additive for animal feed as produced by this method has a lower lysine content due to the existence of other substances and impurities as included in the fermentation broth.

Another limiting factor for lysine content in the granulation process occurs due to the natural hygroscopy of lysine. More specifically, the formulation of lysine has two amine radicals, which makes it become very hygroscopic, i.e. lysine has high capacity/facility to absorb water. More objectively, these two amine radicals in concentrated form tend to be deprotonated only under basic pH, and for this reason liquid concentrated of lysine with more than 700 g/l of purified lysine have pH above of 10.

According to the current state of the art, with the purpose to improve the properties of granulated lysine, a process is known, e. g. by the patent documents EP 1424012 and U.S. Pat. No. 8,916,213, for producing additives for animal feed, by adding to the fermentation broth a concentrated lysine liquid, thus increasing the concentration in the fermentation broth before starting the granulation process.

Despite being theoretically advantageous, considering that it was possible to increase lysine concentration in the final product, in practice, and especially from the commercial point of view, it has been shown to be unfeasible and impractical due to the other subsidiaries characteristics of this final product. More specifically, it is possible to conclude that, by adding and mixing the concentrate lysine liquid to the fermentation broth containing ammonium ions, a very common substance in fermentation media for being used as the main source of nitrogen for the synthesis of the lysine molecule, it provided strong release of ammonia gas, especially in the final product.

As the person skilled in the art may conclude, ammonia gas has a very strong odor causing serious inconveniences, from discomfort among employees while handling it to animal aversion. This means that the odor caused by ammonia gas causes said granulated lysine to be commercially unfeasible or unacceptable as a final product.

Another problem identified at the time of mixing the concentrate lysine liquid with the fermentation broth refers to harmful formulations due to the content of ammoniacal nitrogen which is not consumed by animals, thus masking the total nitrogen content consumed by animals when fed with feed supplemented with additives of this nature.

More particularly regard the high nitrogen content as well as ammonium in the fermentation broth, EP 1813677, CN 104250659 and CN 103215286 are known, which show fermentation processes whose nitrogen and ammoniacal concentration are controlled at high levels in order to guarantee the high production of lysine, i.e., they guarantee that the bacteria will remain working without problems due to the absence of nitrogen until the end of consumption of the carbon source, at which point the fermentation step is interrupted, thus obtaining a fermentation broth with high lysine content but also with a high amount of ammonium and nitrogen, which ends up affecting in its applicability as animal feed, as highlighted above in relation to the odor generated by the ammonia gas release and the effective nitrogen concentration of nitrogen consumed by animals.

Therefore, although these prior art documents reveal processes capable of producing a fermentation broth with high lysine content, its applicability is affected by the nitrogen and ammonium content.

Another inconvenience of the processes for manufacturing lysine-based additives for animal feed as known in the state of the art refers to the excessive use of acids, which are widely used to handle pH of the solutions, thus promoting the concentration and release of lysine. As it should be generally known in the art, and also shown in the document EP 13312220, the excessive use of acid may affect the quality and content of lysine in the final product, besides bringing in costs for the production of additives consequently affecting the final value of the product.

Therefore, as we can see, lysine-based additives for animal feed as known in the state of the art disclose restrictions and inconveniences which may be enhanced and/or solved with the processes and methods as currently used, thus disclosing problems affecting the quality and physical-chemical properties of final products, particularly concerning lysine content and concentration, but also by the high ammonium content caused by the control on high nitrogen content levels in the fermentation step. More specifically, it is possible to note that the state of the art lacks low-cost practical and functional solutions being able to offer a commercially attractive, acceptable and feasible product, which is especially rich in lysine content and has low ammonium content.

### BRIEF SUMMARY OF THE OBJECTIVES OF THE INVENTION

In view of the foregoing, it is one objective of the present invention obtain an alkali granulated additive for animal feed having pH above the conventional one and a substantially high lysine concentration, but having a nearly imperceptible content of ammonium, due to the controlled reduction of the content of ammoniacal nitrogen during the final fermentation step, and said additive is obtained by a new production process, especially developed to simply and efficiently solve the problems and inconveniences as disclosed by the state of the art.

More specifically, it is one of the objectives of the present invention provides an alkali granulated additive for animal feed comprising the following composition:
pH between 8 and 10
molar relation following the condition:

$$\frac{[ {NH}_{4}^{+} ]}{[ L Lys ]} < {10}^{- 2}$$

lysine content between 70-85% by weight.

Under these conditions, it is an objective of the invention produces a granulated additive for animal feed having, besides high lysine concentration and low ammonium content, low hygroscopicity and pH above the conventional due to the low use of inorganic acids and the control of nitrogen content in very low levels during a fermentation step.

It is also an objective of the invention provides a process for manufacturing alkali granulated additives for animal feed which characteristics have been designed and developed to substantially simplify the production lines for additives for lysine-based animal feeds, but especially to obtain considerable reduction in costs involved for the production without negatively affecting physical and chemical properties of said additives. In this context, it should highlight that the inventors have developed a process enabling to substantially reduce the use of acids and chemicals, thus reaching an additive with a very high lysine concentration.

Finally, the invention also refers to an alkali granulated additive for animal feed with pH between about 8-10, with a high lysine concentration, also having a very low ammonium content. In other words, it is an objective of the invention provides, at a relatively reduced cost, a granulated additive for animal feed with high lysine concentration and low ability of ammonia gas emission.

### DESCRIPTION OF EMBODIMENTS OF THE INVENTION

In accordance to the above information, a few examples of preferred and possible embodiments of the invention will be disclosed in more detail below, but it is important to highlight that this is a mere example and not a restrictive disclosure, since the granulated additive for animal feed and said processes for its production, objects of the invention, may present different details and technical and constructive characteristics, without affecting the scope of protection as defined by the attached claims.

Therefore, the alkali granulated additive for animal feed of the invention comprises the following composition:
pH between 8 and 10
molar relation following the following below:

$$\frac{[ {NH}_{4}^{+} ]}{[ L Lys ]} < {10}^{- 2}$$

lysine content between 70 and 85% by weight.

According to the invention, the alkali granulated additive for animal feed manages to have pH between 8 and 10 due to the considerable reduction in use of inorganic acids and chemicals during the production process, thus increasing lysine concentration, previously occupied by other ions, providing the final product with a high lysine concentration.

To allow such a reduction in the use of inorganic acids, consequently providing to obtain a product with high pH, high concentration of lysine and also low hygroscopicity, the inventors have developed a process to control the amount of ammonium ions during the fermentation and lysine formation step. Therefore, it has become possible to obtain a product with very low content of ammonium, consequently reducing odors caused by the emission of ammonia gas which is undesirable for the final commercialization of additives for animal feed.

More specifically, the alkali granulated additive for animal feed of the invention may be obtained by a process wherein feeding, during the fermentation step, occurs with the control of concentration levels for ions of ammonium in the solution by monitoring the content of ammoniacal nitrogen (AN) which in turn is preferably obtained by the so-called Kjeldahl or equivalent method.

In this fermentation step, culture medium is formed, wherein the fermentation of a biomass occurs by means of microorganisms and, for that purpose, they are fed in a controlled way with carbon under the form of sugars (such as glucose, sucrose, etc.), nitrogen by introducing ammonium sulfate (NH₄SO₄), ammonia (NH₃) or urea, besides other components constituted by vitamins and minerals. According to the invention, this step is essential to obtain alkali granulated additive for animal feed rich in lysine which is, at the same time, free from the odor caused by ammonia gas.

More specifically, during this step, to avoid any kind of restriction or limitation to the synthesis of lysine by microorganisms, precise balance is required between the amount of carbon and nitrogen available in the fermentation broth. This is because microorganisms only produce and release lysine in the presence of carbon, but the low amount of nitrogen eventually stresses microorganisms, thus limiting their productivity in lysine release. For this reason, appropriate and precise control of the quantity of carbon and nitrogen is required.

Such control over the amount of nitrogen according to the amount of carbon is monitored by the content of ammoniacal nitrogen (AN) along the processing and fermentation of the culture medium, which is reduced according to the consumption of carbon and release of lysine by microorganisms. When said content of ammoniacal nitrogen (AN) reaches an amount of about 50 mg/l, a solution of ammonium sulfate with a concentration between 200 g/l and 450 g/l is introduced to increase the content of ammoniacal nitrogen (AN) to about 100 mg/l, which, as the time passes, falls again to about 50 mg/l and a new feed of the ammonium sulfate solution is performed so to keep the culture medium with the content of ammoniacal nitrogen (AN) in around 100 mg/l until it is close to the end of the carbon source. In this occasion, the monitoring of said content of ammoniacal nitrogen (AN) is sustained until reaching values between about 50 mg/l and 10 mg/l which will be controlled to be remained within that range until the complete closure of carbon source, at which point the fermentation step is terminated.

Therefore, at the end of this step, when the source of carbon in the culture medium is exhausted, it is obtained a final fermented broth which content of ammoniacal nitrogen (AN) is between 50 mg/l and 10 mg/l, and having a lysine content between 60-85%, thus being able to present small variations caused by the raw material forming the biomass and also the strains of lysine-producing microorganisms. Furthermore, said final fermented broth containing lysine has a molar relation following the condition below:

$$\frac{[ {NH}_{4}^{+} ]}{[ L Lys ]} < {10}^{- 2}$$

Under this condition, it is possible to verify that the concentration of ammonium ions in the fermented broth is practically zero, nearly imperceptible, thus enabling to obtain and produce the alkali granulated additive of the invention, which the properties inhibit the emission of ammonia gas, so to reach a very desirable product to manufacturers, and also to animals.

Giving continuity to the process for producing alkali granulated additive for animal feed, according to the objectives of the invention, the final fermented broth, resulting from the fermentation step in fermentation tanks, may then be processed by granulation, be it conventional or in steps.

As the person skilled in the art should appreciate, e. g. from the document U.S. Pat. No. 7,883,878, conventional granulation also enables to provide a mixture of said fermented broth containing lysine with the so-called pure, purified or concentrated lysine obtained by purification and/or evaporation of a fermented broth containing lysine usually performed by ionic exchange processes, thus allowing to obtain a liquid with very high lysine concentration.

By mixing said purified lysine in the fermented broth with high lysine content and low ammonium content, we reach a solution which may then be used to perform the conventional granulation process, wherein said mixed solution of the fermented broth containing purified lysine may be used to spray a particle, a grain or an inert solid, creating a lysine-containing layer which is dried, and said spraying and drying of that solution mixed over the inert particle, grain or inert solid is subsequently repeated a few times, thus creating the alkali granulated additive for animal feed with high lysine concentration and very low ammonium content. This granulation step may be performed by means of methods and processes already available in the state of the art, as the person skilled in the art should appreciate.

Alternatively, it would also be possible to promote granulation which steps as commented above occur in a relatively simultaneous way, wherein, at the same time, original grains are formed by the final fermented broth and the purified lysine, and these grains are subsequently sprayed and covered by the purified lysine and final fermented broth layers. Under this situation, a kind of agglomeration of grains formed by the fermented broth which are inter-related to the purified lysine may occur, thus forming grains which are very rich in lysine.

It should highlight that, although the spraying method to form covering layers for the original grains is mentioned above, said covering may be performed by other forms and methods as known by the state of the art, liable for application with no damage to the final result.

According to one of the possible embodiments of the invention, as the person skilled in the art should appreciate, said liquid formed by the purified/concentrated lysine may be generated and provided by any known method and process. Optionally, if it is interest to the manufacturers, said purified lysine may be a product which origin is the same of the final fermented broth as shown above, containing high concentration of lysine and very low content of ammonium. Therefore, the final fermented broth with lysine may be divided, wherein one of the parts will be processed to obtain purified lysine.

At the end, the process object of the invention enables to obtain a granulated lysine-based additive for animal feed which pH is between 8-10 and which, besides containing a lysine concentration between 70% and 85% by weight, has a practically negligible content of ammonium ions which molar relation is within the following condition:

$$\frac{[ {NH}_{4}^{+} ]}{[ L Lys ]} < {10}^{- 2}$$

Therefore, it is possible to conclude that the alkali granulated additive object of the invention comprises peculiar properties offering lysine-rich nutrition for animal feed, i.e. containing a substantial amount of lysine, but they mainly disclose substantially reduced costs in comparison with equivalent processes, such as those used for the production of conventional lysine-HCl.

Therefore, it is possible to state that the alkali granulated additive for animal feed of the invention has a similar concentration to Lysine-HCl, but without its high costs and complex processing. Furthermore, at the same time, it discloses a simplified production process which is more economic and similar to the process for the production of granulated lysine. In other words, the lysine-based alkali granulated additive for animal feed, object of the invention, simply and effectively brings together the benefit aspects of the most traditional processes as known in the state of the art, consequently with higher profitability and productivity.

### ILLUSTRATIVE EXAMPLES OF THE INVENTION

### Example 1

As a mere example of a practical embodiment of the alkali granulated additive for animal feed, the process for obtaining it is comprised by the fermentation step in a fermentation tank, which the result is the final fermented broth containing lysine and low content of ammonium ions. For that purpose, the culture medium is constituted by: 65 g/l of sucrose, 1.0 g/l of KH₂PO_{4, 30} g/l of ammonium sulfate; 0.5 g/l of MgSO₄; 5 mg/l of MnSO₄; 5 mg/l of FeSO₄; 200 μg/l of biotin, 200 μg/l thiamine and 20 μ/l of soy hydrolysate. Such culture medium is then sterilized at 121° C. for about 20 minutes.

By using aseptic inoculation techniques, strains of lysine-producing microorganisms (such as Corynebacterium, Escherichia coli) are inoculated in a pressurized fermentation tank at between 0.3 and 0.5 kgf/cm² with an already sterilized medium to avoid contamination.

The temperature in the fermentation tank is controlled between 30 and 36° C., preferably 32° C., and pH is adjusted with ammonia gas NH₃ between 6 and 8, more preferably 7.

From five hours of fermentation, the collection of samples at each five hours starts for the analysis of optical density (OD) and the concentration of ammoniacal nitrogen (AN). As a consequence of the relation between the content of ammoniacal nitrogen and the ammonium ion, it is possible to determine the concentration of ammonium ions in the solution. The optical density (OD) is measured by a spectrophotometer at 560 nm and ammoniacal nitrogen is defined by the Kieldahl method.

After reaching between 15 and 20 hours of fermentation, the concentration of ammoniacal nitrogen (AN) falls to levels between 50 mg/l and 10 mg/l. An ammonium sulfate solution with concentration between 200 g/l and 450 g/l is then introduced to keep the content of ammoniacal nitrogen (AN) at 100 mg/l, awaiting to fall again to 50 mg/l, and then successively until it is close to the end of the carbon source, at which there is a controlled reduction of the content of ammoniacal nitrogen (AN) from 100 mg/L to amounts between 50 mg/L and 10 mg/L that will be maintained under these conditions until the end of the carbon source consumption.

Upon the end of the carbon source, when microorganisms interrupt the release of lysine, the fermentation step ends, resulting in a final fermented broth with lysine and ammoniacal nitrogen content between about 50 mg/l and 10 mg/l.

### Example 2

Giving continuity to Example 1, said fermented broth from the previous example is then mixed to concentrate lysine as provided by a process such as ultrafiltration and ionic exchange, so to reach a solution under concentration of 450 g/l of L-lysine.

Said solution is then concentrated in a rotavapor model IKA RV 8V at 60° C. until the concentration of total solids is 40%. The concentrated liquid is then granulated in a pilot granulator (GR Engineering, Fluid Bed Spray Dryer). In this pilot granulated, the inlet temperature is about 170° C., outlet temperature is about 60-70° C. and spray pressure is about 1.5 bar. At the end, a granulated product is obtained at 79% dry base lysine.

Finally, considering all the above, it is important to make it clear that the single purpose of the present description is to define as an example preferred embodiments of the process for manufacturing alkali granulated additives for animal feed with high lysine concentration and low ammonium content, as well as the alkali granulated additive for animal feed of the present invention itself. Therefore, as the person skilled in the art will well understand, numerous modifications, variations and combinations of embodiments of the elements performing the same function substantially in the same way to reach the same results should be included in the scope of protection as defined by the attached claims.

1-8. (canceled)

9. A method comprising:
fermenting, inside a fermentation tank, a culture medium including a carbon source and a nitrogen source to produce lysine, the fermentation including:
monitoring the nitrogen source by measuring a concentration of ammoniacal nitrogen within the culture medium,
maintaining the concentration of ammoniacal nitrogen at about 100 mg of ammoniacal nitrogen per liter of the culture medium by introducing, in response to the concentration of ammoniacal nitrogen approaching a predetermined level, a solution to the culture medium, the solution being configured to increase the concentration of ammoniacal nitrogen to about 100 mg of ammoniacal nitrogen per liter of the culture medium, and
controlling reduction of the concentration of ammoniacal nitrogen to between 50 mg of ammoniacal nitrogen per liter of the culture medium and 10 mg of ammoniacal nitrogen per liter of culture medium until an end of the carbon source to thereby produce a fermented broth; and
producing, from the fermented broth, an alkali granulated additive for animal feed having a pH between 8 and 10, the alkali granulated additive including:
lysine in a first amount between 70% and 85% by weight on a basis of a weight of the alkali granulated additive, and
ammonia in a second amount being less than 10⁻² mols of ammonia per mol of lysine.

10. The method of claim 9, wherein the solution is an ammonium sulfate solution with a concentration between 200 g ammonium sulfate per liter of solution and 450 g ammonium sulfate per liter of solution, and the predetermined level is 50 mg of ammoniacal nitrogen per liter of the culture medium.

11. The method of claim 9, wherein the fermented broth includes ammoniacal nitrogen between 50 mg of ammoniacal nitrogen per liter of the fermented broth and 10 mg of ammoniacal nitrogen per liter of fermented broth, lysine between 60% and 85% by weight on a basis of a weight of the fermented broth, and a fermented amount of ammonia less than 10⁻² mols of ammonia per mol of lysine.

12. The method of claim 9, wherein producing the alkali granulated additive from the fermented broth includes a granulation procedure.

13. The method of claim 9, wherein the fermented broth is mixed with a concentrated solution of lysine to produce a mixed solution.

14. The method of claim 13, wherein the concentrated solution of lysine is produced using at least one of purification of a fermented solution and evaporation of a fermented solution.

15. The method of claim 14, wherein producing the alkali granulated additive includes:
spraying the mixed solution over a particle, a grain, or an inert solid to produce a coating;
drying the coating to thereby produce a layer; and
repeating the spraying and the drying to form a plurality of covering layers on the particle.

16. An alkali granulated additive for animal feed having a pH between 8 and 10, the alkali granulated additive including lysine in a first amount between 70% and 85% by weight on a basis of a weight of the alkali granulated additive and ammonia in a second amount being less than 10⁻² mols of ammonia per mol of lysine.
